(19) United States
(12) Patent Application Publication
(10) Pub. No.: US 2020/0250593 A1
(43) Pub. Date: Aug. 6, 2020

(54) YIELD ESTIMATION IN THE CULTIVATION OF CROP PLANTS

(71) Applicant: BASF Agro Trademarks GmbH, Ludwigshafen (DE)

(72) Inventors: Ole Peters, Langenfeld (DE); Gang Zhao, Langenfeld (DE); Holger Hoffmann, Langenfeld (DE); Eva Hill, Langenfeld (DE); Ahmed Karim Dhaouadi, Langenfeld (DE); Christian Bitter, Langenfeld (DE); Fabian Johannes Schaefer, Langenfeld (DE)

(21) Appl. No.: 16/756,317

(22) PCT Filed: Oct. 24, 2018
(86) PCT No.: PCT/EP2018/079132

§ 371 Date: Apr. 15, 2020

(30) Foreign Application Priority Data
Oct. 26, 2017 (EP) 17198464.4
Aug. 22, 2018 (EP) PCT/EP2018/072662

(51) Int. Cl. G06Q 10/04; G06Q 50/02; G06Q 10/06; A01D 91/00; G01W 1/10; G16B 5/00

(52) U.S. Cl. CPC G06Q 10/04; G06Q 50/02; G16B 5/00; A01D 91/00; G01W 1/10; G06Q 10/0631

(57) ABSTRACT

The present invention relates to the technical field of the growing of crop plants, especially the creation of forecasts of the expected yield.

The present invention relates to the technical field of the growing of crop plants, especially the creation of forecasts of the expected yield.

The yield of the crop plant being grown is determined by a multitude of parameters. Some of these can be influenced by a farmer, for example soil cultivation, the type, date and density of sowing, the implementation of measures to control harmful organisms, the deployment of nutrients, irrigation and the date of harvesting. Other parameters such as the weather can barely be influenced.

It would be advantageous for the farmer to know what yield he can expect at the end of a growing period. It would also be favorable to know how agricultural measures will affect the yield.

The present invention provides such information for a farmer.

The invention firstly provides a method, preferably of ascertaining yields to be expected in the growing of crop plants with the aid of a computer system, for instance a server, especially a server and a local or mobile computer system, comprising the steps of

(A) identifying a field in which crop plants are being grown or are to be grown, preferably with provision of position data, especially geocoordinates,

(B) forecasting a weather progression for the field for the upcoming or ongoing growing period for the crop plants until the planned harvest taking account of the weather progression to date, preferably with a seamless transition of the weather progression to date and the forecast weather progression,

(C) forecasting for the occurrence of one or more harmful organisms in the field for the forecast weather progression,

(D) ascertaining agricultural measures for the upcoming or ongoing growing period of the crop plants until the planned harvest, preferably with provision of measure data that at least partly predetermine agricultural measures for the upcoming or ongoing growing period,

(E) calculating the yields to be expected in the growing of the crop plants assuming that the forecasts from steps (B) and (C) are correct and the measures ascertained in step (D) are implemented,

(F) providing or displaying the yields to be expected,

(G) repeatedly performing steps (B), (C), (D), (E) and (F) taking account of the real progression of the weather up to the respective juncture of performance of the steps, the harmful organisms that have actually occurred and the measures that have actually been implemented, preferably with provision for step (B) of weather data relating to an actual or existing progression of the weather and, for steps (C), (D), (E), recorded field-specific data, especially harmful organism data relating to harmful organisms that have actually occurred, growth data relating to the real progression of the growth that has actually occurred and/or measure data relating to measures that have actually been implemented, preferably with forecasting of at least two different weather progressions in step (B), and performance of steps (C), (D), (E) for each of the at least two different weather progressions.

The present invention further provides a computer system, preferably for ascertaining yields to be expected in the growing of crop plants, comprising

(A) means of identifying a field in which crop plants are being grown or are to be grown, preferably with provision of position data, especially geocoordinates, or a recording module configured to provide position data, especially geocoordinates,

(B) means of providing a forecast of a weather progression for the field for the upcoming or ongoing growing period of the crop plants until the planned harvest taking account of the weather progression to date, preferably with a seamless transition of the weather progression to date and the forecast weather progression, or a weather module configured to provide a forecast of a weather progression for the field for the upcoming or ongoing growth period of the crop plants until the planned harvest taking account of the weather profile to date, preferably with a seamless transition of the weather progression to date and the forecast weather progression,

(C) means of providing a forecast for the occurrence of one or more harmful organisms in the field for the forecast weather progression, or a harmful organism module configured to provide a forecast for the occurrence of one or more harmful organisms in the field for the forecast weather progression,

(D) means of identifying agricultural measures for the upcoming or ongoing growth period of the crop plants until the planned harvest, preferably with provision of measure data that at least partly predetermine agricultural measures for the upcoming or ongoing growth period, or a measure module configured to identify agricultural measures for the upcoming or ongoing growth period of the crop plants until the planned harvest, preferably with provision of measure data that partly predetermine agricultural measures for the upcoming or ongoing growth period,

(E) means of calculating the yields to be expected in the growing of the crop plants assuming that the forecasts from steps (B) and (C) are correct and the measures ascertained in step (D) are implemented, or a yield module configured to calculate yields to be expected in the growing of the crop plants assuming that the forecasts from steps (B) and (C) are correct and the measures ascertained in step (D) are implemented,

(F) means of displaying or providing the yields to be expected, or an interface configured to display or to provide yields to be expected, wherein the computer system is configured such that it repeatedly performs steps (B), (C), (D), (E) and (F) taking account of the real progression of the weather up to the respective juncture of performance of the steps, the harmful organisms that have actually occurred and the measures that have actually been implemented,

preferably with provision for step (B) of weather data relating to an actual or existing progression of the weather and, for steps (C), (D), (E), recorded field-specific data, especially harmful organism data relating to harmful organisms that have actually occurred, growth data relating to the real progression of the growth that has actually occurred and/or measure data relating to measures that have actually been implemented, preferably with forecasting of at least two different weather progressions in step (B), and performance of steps (C), (D), (E) for each of the at least two different weather progressions.

The present invention further provides a computer program product, preferably for ascertaining yields to be expected in the growing of crop plants, comprising a computer-readable data storage means and program code stored on the data storage means, and which, when executed on a computer system, causes the computer system to execute the following steps:

(A) ascertaining a field in which crop plants are being grown or are to be grown, preferably with provision of position data, especially geocoordinates,

(B) ascertaining a weather progression for the field for the upcoming or ongoing growing period for the crop plants until the planned harvest taking account of the weather progression to date, preferably with a seamless transition of the weather progression to date and the forecast weather progression,

(C) ascertaining a forecast for the occurrence of one or more harmful organisms in the field for the forecast weather progression,

(D) ascertaining agricultural measures for the upcoming or ongoing growing period of the crop plants until the planned harvest, preferably with provision of measure data that at least partly predetermine agricultural measures for the upcoming or ongoing growing period,

(E) calculating the yields to be expected in the growing of the crop plants assuming that the forecasts from steps (B) and (C) are correct and the measures ascertained in step (D) are implemented,

(F) providing or displaying the yields to be expected,

(G) repeatedly performing steps (B), (C), (D), (E) and (F) taking account of the real progression of the weather up to the respective juncture of performance of the steps, the harmful organisms that have actually occurred and the measures that have actually been implemented, preferably with provision for step (B) of weather data relating to an actual or existing progression of the weather and, for steps (C), (D), (E), recorded field-specific data, especially harmful organism data relating to harmful organisms that have actually occurred, growth data relating to the real progression of the growth that has actually occurred and/or measure data relating to measures that have actually been implemented,

preferably with forecasting of at least two different weather progressions in step (B), and performance of steps (C), (D), (E) for each of the at least two different weather progressions.

The invention is explained in detail hereinafter without distinguishing between the subjects of the invention (method, computer system, computer program product). Instead, the elucidations that follow are intended to be analogously applicable to all subjects of the invention, irrespective of their context (method, computer system, computer program product).

The method of the invention serves to assist a farmer in the growing of crop plants in a field.

The term “field” is understood to mean a spatially delimitable region of the surface of the Earth which is in agricultural use by planting of crop plants in such a field, supplying them with nutrients and harvesting them.

The term “crop plant” is understood to mean a plant that is purposely grown as a useful or ornamental plant through human intervention.

(A) Identifying a Field in which Crop Plants are being Grown or are to be Grown

In a first step, the field in which crop plants are being grown or are to be grown, and which is considered in detail in the course of the method of the invention, is identified.

Typically, the identification is effected using geocoordinates that unambiguously determine the location of the field. The method of the invention is typically executed with the aid of a computer program installed on a computer system. Typically, the geocoordinates of the field are therefore transferred into the computer program. For example, a user of the computer program could input the geocoordinates via a keyboard. It is also conceivable that the user of the computer program views geographic maps on a computer screen and marks the boundaries of the field under consideration on such a map, for example with a computer mouse.

The identification of the field accordingly fixes the region of the earth's surface that is considered in the further course of the method of the invention.

(B) Forecasting a Weather Progression

In a further step, a weather progression is forecast for the upcoming or ongoing growing period for the crop plants until the planned harvest taking account of the weather progression to date, preferably with a seamless transition of the weather progression to date and the forecast weather progression.

The aim of the forecast of the weather progression is to forecast the distribution and corresponding probabilities of weather events for the upcoming or ongoing growth period with maximum precision.

As is well known, the weather can be forecast comparatively accurately for the next few days, for example up to nine days, whereas weather forecasts for a date in a few weeks or months, for example greater than nine days, in the future are comparatively inexact. For periods in which the weather can be forecast only inexactly, therefore, historic weather data are of good suitability, in order to use trends that have frequently been observed in the past few years as a basis for the forecast of the future weather.

Weather forecasts for the near future (for example one day up to about one week or up to about 9 days) can be sourced, for example, from a multitude of commercial suppliers.

For further into the future (for example more than one week or more than 9 days) within the growth period, preference is given to using seasonal weather forecasts. These forecasts may be based here, for example, on global, regional and globally-regionally coupled dynamic circulation models and/or long-term statistics of historic weather data and/or a dynamic projection (circulation model) of individual climate variables combined with stochastic weather simulation of further variables and/or purely stochastic weather simulations.

In particular, seasonal forecasts may be provided by commercial suppliers and/or research institutes.

The decision as to what kind of seasonal forecast is taken depends on the forecast quality of the model. For this purpose, it is possible to use an index, for example the Brier score. Below a particular limit below which the added value of the modeled weather forecast is insignificant with respect to long-term climate statistics, preference is given to seasonal weather forecasts based on long-term climate statistics.

It is conceivable that multiple forecasts, called projections, are created. It is conceivable that, using historic weather data, a typical (for example most probable) or average weather progression (an average of the weather progressions in a defined period of time, for example the last three, four, five, six, seven, eight, nine, ten years) is ascertained. It is conceivable that, on the basis of the recent past, particular seasonal weather forecasts that seem more likely than others are ascertained.

It is conceivable that, in addition, historic weather data are used to make a forecast for a weather progression that is comparatively favorable—from an agricultural point of view—and/or a weather progression that is comparatively unfavorable.

In a preferred embodiment, multiple weather forecasts are created, which preferably cover the spectrum of the weather progressions as have occurred in the past few years. In a preferred embodiment, a probability is also ascertained and reported for the occurrence of each weather progression, such that the weather progressions can be compared with one another.

The different weather progressions (historic, near-future forecast, seasonal forecast, projections) are combined in seamless time sequences (“seamless prediction”).

(C) Forecast for the Occurrence of One or More Harmful Organisms

For every forecast weather progression, in a further step of the method of the invention, a forecast is made for the occurrence of one or more harmful organisms.

Preferably, the forecast ascertains risks of infestation for one or more harmful organisms.

A “harmful organism” is understood to mean an organism that can appear in the growing of crop plants and can damage the crop plant, adversely affect the harvest of the crop plant or compete with the crop plant for natural resources. Examples of such harmful organisms are weed plants, weed grasses, animal pests, for example beetles, caterpillars and worms, fungi and pathogens (e.g. bacteria and viruses). Even though viruses are not among the organisms from a biological point of view, they shall nevertheless be covered here by the term “harmful organism”. Specific examples of harmful organisms are: Septoria Trititici (https://gd.eppo.int/taxon/SEPTTR), Erysiphe graminis (https://gd.eppo.int/taxon/ERYSGR), Puccinia recondite (https://gd.eppo.int/taxon/PUCCRE), Pyrenophora tritici-repentis or. Drechslera tritici-repentis (https://gd.eppo.int/taxon/PYRNTR) and Fusarium spp. (https://gd.eppo.int/taxon/FUSASP) on winter wheat in central Europe.

The term “weed plant” (plural: weed plants) is understood to mean plants from spontaneous accompanying vegetation (segetal flora) in crop plant crops, grassland or gardens that are not deliberately planted there and develop, for example, from the seed potential in the soil or by aerial transmission. The term is not limited to weeds in the actual sense, but also includes grasses, ferns, mosses or woody plants.

In the crop protection sector, the term “weed grass” (plural: weed grasses) is frequently also utilized in order to illustrate a delimitation from the herbaceous plants. In the present text, the term “weed” is used as an umbrella term that is intended to include the term “weed grass”.

For the forecasting of the occurrence of one or more harmful organisms, it is possible, for example, to use prediction models described in the prior art. The commercially available decision support system “expert”, for prediction, uses data relating to the crop plants being grown or to be grown (stage of development, growth conditions, crop protection measures), relating to weather conditions (temperature, hours of sunshine, wind speed, precipitation) and relating to the known harmful organisms/diseases (limits of economic viability, seedling/disease pressure) and calculates a risk of infestation on the basis of these data (Newe M., Meier H., Johnen A., Volk T.: proPlant expert.com—an online consultation system on crop protection in cereals, rape, potatoes and sugarbeet. EPPO Bulletin 2003, 33, 443-449; Johnen A., Williams I. H., Nilsson C., Klukowski Z., Luik A., Ulber B.: The proPlant Decision Support System: Phenological Models for the Major Pests of Oilseed Rape and Their Key Parasitoids in Europe, Biocontrol-Based Integrated Management of Oilseed Rape Pests (2010) Ed.: Ingrid H. Williams. Tartu 51014, Estonia. ISBN 978-90-481-3982-8. p. 381-403; www.proPlantexpert.com).

For forecasting of harmful organisms, it is also possible to take account of actual infestations in the past.

Preferably, risks of infestation are ascertained for those harmful organisms that have occurred in the past in the field in question and/or adjacent fields.

The risks of infestation are preferably ascertained in a part-area-specific manner. It is conceivable, for example, that some part-areas of the field, owing to their location, are particularly frequently and/or particularly significantly affected by a harmful organism and/or that the infestation with a harmful organism frequently emanates from one or more defined part-areas.

In a preferred embodiment, for a forecast of the weather progression, one or more digital maps of the field in which the risk of infestation with one or more harmful organisms is drawn in a part-area-specific manner are generated. For example, it is conceivable to generate a series of digital maps for a defined harmful organism, for example one map for every month in the year, and to indicate how high is the risk of infestation of the part-area with the harmful organism in the month in question and with the forecast weather progression by means of color coding on the maps. For example, the color “red” could mean a risk of infestation of greater than 90%, and the color “green” a risk of infestation of less than 10%. Different yellow and orange shades could be used for the range between 10% and 90%. Other/further modes of representation are conceivable.

In a preferred embodiment, an assessment is made for risks of infestation ascertained as to whether or not a damage threshold has been exceeded.

“Damage threshold” is a term from agriculture, forestry and horticulture. It indicates the infestation density with pathogens or diseases or infestation with weeds from which control is economically unviable. Up to this value, extra economic expenditure through control is greater than the harvest failure of which there is a risk. If the infestation or weed pressure exceeds this value, the control costs are at least compensated for by the extra yield to be expected.

According to the nature of a harmful organism or disease, the damage threshold may be very different. In the case of harmful organisms or diseases that can be controlled only with high expenditure and adverse accompanying effects on further production, the damage threshold can be very high. If, however, even a small infestation can become a propagation source that threatens to destroy the entire production, the damage threshold may be very low.

There are many examples in the prior art relating to the ascertaining of damage thresholds (see, for example, Claus M. Brodersen: Informationen in Schadschwellenmodellen [Information in Damage Threshold Models], Reports from the GIL [German Society for Computer Science in Agriculture, Forestry and Food Science], volume 7, pages 26 to 36, http://www.gil-net.de/Publikationen/7_26.pdf).

(D) Ascertaining Agricultural Measures

In a further step, agricultural measures are ascertained for the upcoming or ongoing growing period of the crop plants until the planned harvest.

The term “agricultural measure” is understood to mean any measure in the field for crop plants that is necessary or economically viable and/or environmentally advisable in order to obtain a plant product. Examples of agricultural measures are: soil cultivation (e.g. ploughing), deploying the seed (sowing), irrigation, application of growth regulators, control of weed plants/weed grasses, deployment of nutrients (for example by fertilizing), control of harmful organisms, harvesting.

The agricultural measures are preferably measures of chemical crop management (application of crop protection products or growth regulators), especially of reducing the forecast risk of infestation with a harmful organism. The measures are ascertained especially by the selection of a suitable crop protection product, the fixing of dates when the crop protection product should be applied, and fixing of the amount of crop protection product to be applied. The measures are preferably ascertained in a part-area-specific manner.

The term “crop protection product” is understood to mean a composition that serves to protect plants or plant products from harmful organisms or to prevent their effect, to destroy unwanted plants or plant parts, to inhibit unwanted growth of plants or to prevent such growth and/or to influence the life processes of plants in a different manner than nutrients. Examples of crop protection products are herbicides, fungicides and pesticides (for example insecticides).

Preference is given to ascertaining those measures that have a maximum cost/benefit ratio.

The ascertaining of measures preferably takes account of legal aspects and environmental protection aspects. For example, it is conceivable that a selected crop protection product may be applied only at particular dates and/or in particular maximum amounts. These and similar restrictions are preferably taken into account in the ascertaining of the measures.

The measures can be ascertained, for example, on the basis of the crop plants being grown or to be grown. It is conceivable, for example, that a user inputs information about the crop plants being grown or to be grown into the computer system of the invention, for example the name of the species, the sowing date and the like. The computer system then ascertains, for example on the basis of information stored in a database, which measures are required and/or economically viable and/or ecologically advisable in order to achieve a maximum yield. Preferably, the computer system of the invention, on the basis of stored information, determines periods of time in the future in which the measures should sensibly be implemented. The determination of the periods of time may take account of the forecast weather progression and/or the forecast occurrence of harmful organisms. For example, it would not be sensible to implement the harvest of a cereal grown if rain is forecast. Moreover, application of a means of controlling a harmful organism would only be advisable if there is a significant risk of the occurrence of the harmful organism.

(E) Calculation of the Yields to be Expected

In a further step, the yields to be expected when the crop plants are grown under the conditions of the scenarios under consideration are ascertained.

For this purpose, a plant growth model may be used.

The term “plant growth model” is understood to mean a mathematical model that describes the growth of a plant as a function of intrinsic (genetic) and extrinsic (environmental) factors.

Plant growth models exist for a multitude of crop plants. An introduction into the creation of plant growth models is given, for example, by the books i) “Mathematische Modellbildung and Simulation” [Mathematical Modeling and Simulation] by Marco Günther and Kai Velten, published by Wiley-VCH Verlag in October 2014 (ISBN: 978-3-527-41217-4), and ii) “Working with Dynamic Crop Models” by Daniel Wallach, David Makowski, James W. Jones and Francois Brun, published in 2014 in Academic Press (Elsevier), USA.

The plant growth model typically simulates the growth of a crop of crop plants over a defined period of time. It is also conceivable to use a model based on a single plant that simulates the flows of energy and matter in the individual organs of the plant. Mixed models are additionally usable.

The growth of a crop plant is determined not only by the genetic features of the plant but primarily by the local weather conditions that exist over the lifetime of the plant (quantity and spectral distribution of the insolation, temperature profiles, amounts of precipitation, wind input), the condition of the soil and the nutrient supply.

The crop measures that have already been taken and any infestation with harmful organisms that has occurred can also exert an effect on the plant growth and can be taken into account in the growth model.

The plant growth models are generally what are called dynamic process-based models (see “Working with Dynamic Crop Models” by Daniel Wallach, David Makowski, James W. Jones and Francois Brun, published in 2014 in Academic Press (Elsevier), USA), but may also be entirely or partly rule-based or statistical or data-supported/empirical. The models are generally what are called point models. The models here are generally calibrated such that the output reflects the spatial representation of the input. If the input has been ascertained at a point in space or is interpolated or estimated for a point in space, it is generally assumed that the model output is applicable to the whole adjacent field. Application of what are called point models calibrated at the field level to wider, generally rougher scales is known (see, for example, H. Hoffmann et al.: Impact of spatial soil and climate input data aggregation on regional yield simulations, 2016, PLoS ONE 11(4): e0151782.

doi:10.1371/journal.pone.0151782). Application of this so-called point model to multiple points within a field enables part-area-specific modeling here. However, spatial dependences are neglected here, for example in the groundwater budget. On the other hand, there also exist systems for time/space-explicit modeling. Spatial dependences are taken into account here.

Examples of dynamic, process-based plant growth models are Apsim, Lintul, Epic, Hermes, Monica, STICS inter alia.

The following input parameters are preferably included in the modeling: Weather: daily precipitation totals, total radiation, daily minimum and maximum air temperature, and near-ground temperature and ground temperature, wind speed, inter alia.

Soil: soil type, soil texture, soil nature, field capacity, permanent wilting point, organic carbon, mineral nitrogen content, lodging density, van Genuchten parameters, inter alia.

Crop plant: type, variety, variety-specific parameters, for example specific leaf area index, temperature totals, maximum root depth, inter alia.

Crop measures: seed, sowing date, sowing density, sowing depth, fertilizer, fertilizer volume, number of fertilizing dates, fertilizing date, soil cultivation, harvest residues, crop rotation, distance from the field of the same crop last year, irrigation, inter alia.

The forecast of the evolution of the crop plants grown with time is preferably part-area-specific.

The yields to be expected are calculated assuming that the forecasts ascertained beforehand are correct (weather progression, occurrence of harmful organisms) and the agricultural measures ascertained are implemented. It should of course be noted that there can be an interaction between the occurrence of harmful organisms and the agricultural measures. This is because it can be the aim of an agricultural measure to prevent the occurrence of a forecast harmful organism or to reduce the risk. In such a case, the statement “assuming that the forecasts from steps (B) and (C) are correct and the measures ascertained in step (D) are implemented” means that the weather progression occurs as forecast and a risk of the occurrence of harmful organisms does exist as forecast on account of the forecast weather progression, but that the agricultural measures ascertained are implemented and will be successful, which leads to a reduced risk of the occurrence of harmful organisms in relation to the control of harmful organisms (although the risk may also be negligible if the ascertained agricultural has the aim of preventing occurrence of harmful organisms).

The calculation of the yields to be expected can also be made assuming that the agricultural measures ascertained beforehand are not taken. It is conceivable that the user of the computer program product of the invention can study the effect of the measures on the yields to be expected on a computer by, for example, deselecting recommended measures and then the computer program calculates how the yield changes if the measure deselected is not implemented.

Measures are preferably selected and deselected in a part-area-specific manner.

(F) Providing or Displaying the Yields to be Expected

The yields to be expected are displayed to a user on a display device. The display device is typically a screen which is part of the computer system of the invention.

Preferably, the yield to be expected is indicated for individual part-areas and/or the entire field. The display may be graphic-assisted, for example with the aid of bar diagrams or the like.

The user is thus able to view various scenarios on the computer screen and see what yields are the result if a particular forecast weather progression is actually realized and/or what yields are the result if particular measures are taken or not taken.

Preferably, the yields to be expected are displayed in a part-area-specific manner in the form of digital maps on the computer screen.

(G) Repeated Performance of the Steps

In a further step, the steps (B), (C), (D), (E) and (F) mentioned are repeated, taking account of the progression of the weather up to the respective juncture of implementation of the steps, the harmful organisms that have actually occurred and agricultural measures that have actually been implemented.

In the repeated performance, the actual weather progression to date is ascertained. A forecast of the future weather progression is created, which follows seamlessly on from the actual weather progression to date, i.e. there are no discontinuities in the progression of any parameter that describes the weather (temperature, air pressure, air humidity, etc.). For the weather progression ascertained (to date and in the future), the probability of the occurrence of one or more harmful organisms is calculated. Agricultural measures that are to be implemented on the field are ascertained. In the ascertaining of the agricultural measures, it is possible to take account of the weather progression ascertained and/or the forecast harmful organisms. The yields of the crop plants being grown that are to be expected are calculated, it being assumed for the calculation that the future weather progression forecast will actually occur, the risk of the occurrence of forecast harmful organisms actually exists as forecast, and the agricultural measures ascertained are actually implemented and are successful, meaning that the results to be achieved by the measures actually occur. The yields calculated are displayed to the user.

The repetition may be initiated, for example, by the user of the computer system of the invention whenever the user would like to obtain an updated determination of yield.

The computer program product of the invention is preferably configured such that it is automatically updated. Updating means that the weather progression that has actually occurred up to the juncture of the respective update, the harmful organisms that have actually occurred and the measures that have actually been implemented are included in the calculation of yields to be expected. The updating can be effected automatically, for example, whenever the user starts or calls up the computer program. It is alternatively conceivable that the update is effected at a fixed time, for example every day or every week. It is alternatively conceivable that an update is effected at irregular intervals, for example whenever there is a significant deviation of the real conditions from those forecast.

In the event of an update, the steps (B), (C), (D), (E) and (F) detailed above are repeated. Assuming that the user has executed the computer program product of the invention on a first occasion at a first juncture and the yields can be calculated for a forecast weather progression and on the condition that the measures recommended from step (D) are actually taken. At a later, second juncture, the user calls up the computer program product of the invention again. In the intervening period, there was a defined weather progression that affects the plant growth of the crop plants grown and/or the risk of infestation with harmful organisms. The computer program product of the invention ascertains the actual weather progression and adjusts the forecast for the risk of infestation to the actual weather progression. In addition, one or more updated weather forecasts are created and the corresponding risks of infestation are likewise updated. On the basis of the updated risks of infestation, new measures for controlling the harmful organisms are ascertained.

In addition, in a repetition or else in the first run of the steps of the process of the invention, model runs can be adjusted to reality with the aid of further observed state variables. Examples of such an adjustment are the adjusting of the model runs to
the vegetation index (e.g. NDVI) or leaf area index (LAI) observed by means of satellites
infections actually observed in the plant crop (e.g. scorings)
stages of growth actually observed or maturity of the crop
agricultural measures actually taken (fertilization, crop protection, etc.)
further environmental variables observed (e.g. measurements of soil moisture content).

Finally, an updated yield to be expected is calculated and displayed. It is possible here to adjust internal model parameters or calculated state variables.

The method of the invention may be executed entirely or partly on a computer system (for example the computer system of the invention).

A computer system comprises one or more computers. The term “computer” is understood to mean a universally program-controlled machine for information processing. A computer has at least one input unit by means of which the data and control commands can be input (mouse, trackpad, keyboard, scanner, webcam, joystick, microphone, barcode reader etc.), a processing unit comprising working memory and processor with which data and commands are processed, and an output unit in order to transmit data from the system (e.g. screen, printer, loudspeaker etc.). Modern computers are often divided into desktop computers, portable computers, laptops, notebooks, netbooks and tablet computers, and what are called handhelds (e.g. smartphones, smartwatches).

By means of an input unit, a user can select a field for which a yield forecast is to be created. The computer system can provide the user with a digital map. By means of an input unit, for example a computer mouse, the user is able to change the section of the map and zoom into the map or zoom out of the map, such that he is able to display a particular field on the map. In the map, the user is able to select a specific field, for example by drawing field boundaries. It is alternatively conceivable that field boundaries are recognized automatically by means of image analysis and the user can select a recognized field, for example by clicking on it with the mouse.

It is conceivable that the user, by means of an input unit, specifies the crop plants that are being grown (or are to be grown) in the field.

The computer system of the invention may be configured such that it generates weather forecasts itself or sources weather forecasts from a supplier via a connected network (e.g. the Internet).

Preferably, the computer system of the invention is configured such that it sources weather forecasts from a supplier. In such a case, the computer system of the invention comprises a receiving unit for receiving weather forecasts for the specified field or the region including the specified field. The computer system is preferably connected to a network (e.g. the Internet).

By means of a network, the computer system of the invention may also be connected to one or more databases storing information relating to the crop plants being grown/to be grown, for example agricultural measures for the crop plants.

The computer system of the invention may be configured such that it can calculate probabilities of the occurrence of harmful organisms on the basis of the forecast weather progression. In such a case, it is possible to install a prediction model that receives data characterizing the weather progression (for example temperature progressions, amounts of precipitation etc.) as input parameters and outputs probabilities of the occurrence of harmful organisms in the course of the growing phase as output parameters.

It is alternatively conceivable that the computer system of the invention accesses a prediction model via the network in order to obtain and source ascertained risks of infestation.

The computer system of the invention preferably has a unit for calculating yields which may be part of the processing unit. A further part of the yield calculation unit is a plant growth model.

The yield calculation unit uses the weather progression ascertained for the growth period as input parameter in order to calculate plant growth over the growth period by means of the plant growth model. Any forecast harmful organisms and agricultural measures ascertained are likewise taken into account as input parameters. The result is a yield forecast. If multiple weather progressions and/or various agricultural measures have been taken into account, the result is correspondingly multiple yield forecasts.

The computer system has a display device (e.g. a screen) on which it can display the yield forecasts to a user.

### BRIEF DESCRIPTION OF THE FIGURES

Working examples of the invention are detailed in the drawings and elucidated in detail in the descriptions that follow. The figures show:

FIG. 1 an illustrative localized computer system comprising a server, a local computer system, a mobile computer system, an agricultural machine and a satellite system,

FIG. 2 an illustrative method of ascertaining the yields to be expected in the growing of the crop plant with the aid of the localized computer system and especially of the server of FIG. 1,

FIG. 3 an illustrative method of updating the yields to be expected in the growing of the crop plant with the aid of the localized computer system and especially of the server of FIG. 1,

FIG. 4 a further illustrative method of updating the yields to be expected in the growing of the crop plant with the aid of the localized computer system and especially of the server of FIG. 1.

### BRIEF DESCRIPTION OF THE EMBODIMENTS

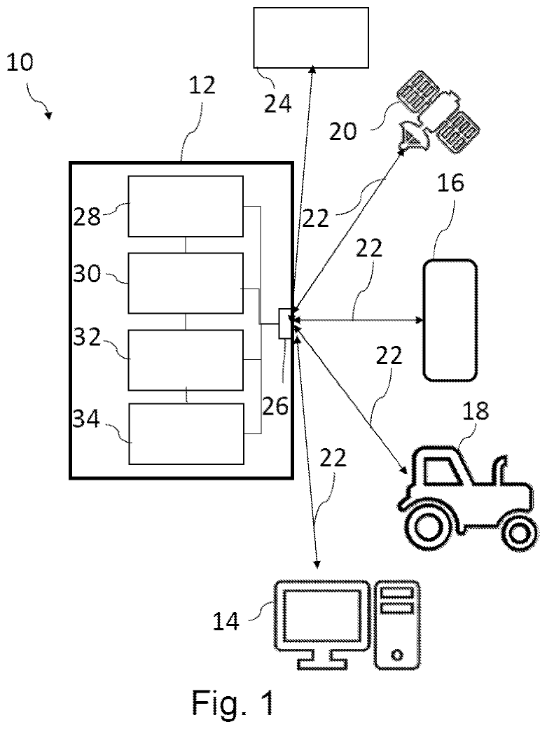

FIG. 1 shows an illustrative localized computer system 10 comprising a server 12, a local computer system 14, a mobile computer system 16, an agricultural machine 18 and a satellite system 20.

The server 12 here may be a cloud server that provides IT infrastructure for storage space, computing power or application software. Local computer systems 14 such as a desktop computer or mobile computer systems 16 such as a smartphone, a drone, a portable digital assistant (PDA), a laptop or a tablet can communicate with the server 12 via a network 22 such as the Internet. In addition, agricultural machines 18 or satellite systems 20 can communicate with the server.

The local computer system 14 can function as a client and may comprise a web-based application that orchestrates communication with the server 12. For example, requests for ascertaining a yield are sent to the server 12 or requested data, such as yields ascertained and scenarios for the ascertaining, are received from the server 12. For instance, the request for ascertaining a yield may comprise position data of the field, time data, field-specific data, especially growth data, harmful organism data or measure data. In addition, the local computer system 14 may serve for visualization of data, for instance the yields ascertained and the assumptions or scenarios that led to the yields ascertained, on a screen.

In an analogous manner, the mobile computer system 16, such as the smartphone, laptop or tablet, can function as client and may comprise a web-based application that orchestrates communication with the server 12. In addition, the mobile computer system 16, such as a smartphone or drone, can be used directly in the field in order to communicate field-specific data to the server 12. For example, a camera in the mobile computer system 16 can be utilized for generation of image data. For instance, local image data of the field can be recorded with the aid of the mobile computer system 16 and transmitted to the server 12 in order to ascertain yield forecasts, for instance. Image and/or object analysis methods can extract growth, infestation or agricultural measures from the image data. The image data can accordingly function as growth data, infestation data and/or measure data in order to ascertain yield forecasts, for instance. In addition, scoring can be recorded with the aid of the mobile computer system 16 and transmitted to the server 12 in order to ascertain yield forecasts, for instance.

In addition, agricultural machines 18 can record agricultural measures via sensors installed therein. For example, an agricultural machine 18 for deployment of seed can record position data of the deployment, type of seed, amount of seed deployed and date of deployment. In an analogous manner, an agricultural machine 18 for deployment of crop protection products can record position data of the deployment, type of crop protection product, amount of crop protection product deployed and date of deployment. In this way, it is possible to record measured data that specify, for example, seeding measures, fertilizing measures, soil cultivation measures, crop protection measures or irrigation measures. The measured data recorded can be transmitted to the server 12 in order to ascertain yield forecasts, for instance.

In addition, measurements can be detected by satellite systems 20 and transmitted to the server 12. For instance, earth observation satellites for remote sensing, on the basis of different measurement techniques, such as LI DAR, RADAR, hyper- or multispectral spectrometry or photography, can record weather data, or field-specific data such as growth data, infestation data and/or measure data. More particularly, it is possible to extract growth data from satellite images, such as the biomass of a field or the leaf area index. Navigation satellites can be utilized for location or for ascertaining of position data. The weather data recorded or the field-specific data recorded can be transmitted to an external database 24 that can be accessed by the server 12, or the weather data or field-specific data recorded can be transmitted directly to the server 12.

In addition, the server 12 may comprise a recording module 26 for sending and receiving data via a network such as the Internet. Via the recording module 26, the server 12 may be connected via a network such as the Internet to further networkable devices 14, 16, 18, 20, such as a desktop computer 14, a smartphone 16, an agricultural machine 18 or a satellite system 20. For instance, field-specific data can be transmitted via the recording module 26 by the mobile computer system 16, the agricultural machine 18 or the satellite system 20.

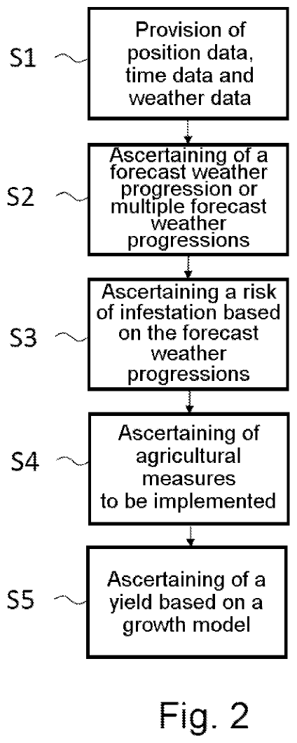

The server 12 is configured to determine the yield to be expected in the field under consideration. For this purpose, the server especially comprises a weather data module 28, a harmful organism module 30, a measure module 32 and a yield module 34. The recording module 26 provides, for example, position data, time data, weather data, field-specific data or historic data. The weather module 28 provides, for example, models for ascertaining the weather progression and ascertains a forecast weather progression, as described in FIGS. 2 to 4. For this purpose, the weather module 28 may be in communication with the recording module 26 that provides corresponding weather data. The harmful organism module 30 provides, for example, models for the occurrence of harmful organisms and ascertains a risk of infestation, as described in FIGS. 2 to 4. For this purpose, the harmful organism module 30 may be in communication with the recording module 26 that provides corresponding harmful organism data. The measure module 32 provides, for example, models for ascertaining agricultural measures and ascertains agricultural measures, as described in FIGS. 2 to 4. For this purpose, the measure module 32 may be in communication with the recording module 26 that provides corresponding measure data. The yield module 34 provides, for example, models for ascertaining the yields to be expected and ascertains yields to be expected, as described in FIGS. 2 to 4. For this purpose, the yield module 34 may be in communication with the recording module 26 that provides corresponding growth data.

FIG. 2 shows an illustrative method of ascertaining the yields to be expected in the growing of the crop plants with the aid of the decentralized computer system 10 and especially with the aid of the server 12 of FIG. 1. The method, as shown in FIG. 2, can be implemented before or at the sowing date. In that case, the current date is before or on the sowing date and the method may especially be utilized for planning the upcoming growth period.

In a first step S1, position data that identify the field and time data that specify the current date and/or a harvesting date are provided. The position data and time data may be generated on a local or mobile computer system 14, 16 and transmitted to the server 12. The current date may specify a predetermined date or the current time, recorded, for instance, by the local or mobile computer system 14, 16. The harvesting date may specify a predetermined date of planned harvesting, or the optimal date of planned harvesting can be ascertained with the aid of a growth model.

In one embodiment, position data, especially geocoordinates, can be recorded with the aid of a mobile computer system 16 comprising a location sensor, such as a GPS sensor. The transmission of the position data from the mobile computer system 16 to the server 12 may be triggered here when the mobile computer system 16 is at the location of the field. Alternatively, position data, especially geocoordinates, can be provided with the aid of an input module, such as a keyboard, a computer mouse or a touch-sensitive screen, of the local or mobile computer system 14, 16. For this purpose, the position data can be transmitted from the local or mobile computer system 14, 16 to the server 12. More particularly, geographic maps, for instance satellite maps, may be provided on the local or mobile computer system 14, 16, in order to specify the field under consideration. The geocoordinates may comprise coordinates of the field boundary or a base coordinate and a field boundary shape associated therewith.

Moreover, in the first step S1, weather data may be provided for the current date and for past dates before the current date. For example, the period of the past dates may relate to the year of the upcoming growth period. The weather data may be data recorded in weather stations relating, for example, to temperature, hours of sunshine, wind speed, precipitation, daily precipitation totals, sunlight totals, daily minimum and maximum air temperature, near-ground temperature, ground temperature. The weather data may be transmitted from weather stations to the server 12 or to an external database 24 that can be accessed by the server. The weather data can be used to ascertain an actual weather progression or a weather progression up to the current date.

In a second step S2, a forecast weather progression for a forecast period can be ascertained at least on the basis of the weather data provided up to the current date or the weather progression to date. It is possible here for the forecast period to comprise a period between the current date and the harvesting date. The forecast period may consist of a period between the current date and the harvesting date. The weather progression can be forecast for the upcoming growing period for the crop plants until the harvesting date taking account of the weather progression to date, preferably with a seamless transition of the weather progression to date and the forecast weather progression. The aim of the forecast of the weather progression is to forecast the distribution and corresponding probabilities of weather events for the upcoming growth period with maximum precision.

As is well known, the weather can be forecast comparatively accurately for the next few days, for example up to nine days, whereas weather forecasts for a date in a few weeks or months, for example greater than nine days, in the future are comparatively inexact. For periods in which the weather can be forecast only inexactly, therefore, historic weather data may therefore be of good suitability, in order to use trends that have frequently been observed in the past few years as a basis for the forecast of the future weather.

Weather forecasts for the near future (for example one day up to about one week or up to about 9 days) can be sourced, for example, from a multitude of commercial suppliers. The forecast weather progression may comprise a short-term forecast of the weather progression from the current date until a first date after the current date or for the near future or a period from the current date until a few days, for instance up to 9 days, after the current date. Such short-term forecast weather progressions from the current date until a first date after the current date may be provided by an external database 24 that can be accessed by the server 12 and transmitted to the server 12 or ascertained on the server 12. For example, short-term forecast weather progressions are ascertained on the basis of dynamic weather models and possibly taking account of the weather progression to date or the weather data at the current date.

Additionally or alternatively, the forecast weather progression may comprise a long-term forecast weather progression until the planned harvesting date from about the first date after the current date until the planned harvesting date, with the long-term weather progression covering the period further into the future or a period from the current date or from the end date, for instance the 9th day, of the short-term forecast weather progression until the harvesting date. For further into the future (for example more than one week or more than 9 days) within the growth period, preference is given to using seasonal weather forecasts. These forecasts may be based on global, regional and globally-regionally coupled dynamic circulation models and/or long-term statistics of historic weather data and/or a dynamic projection (circulation model) of individual climate variables combined with stochastic weather simulation of further variables and/or purely stochastic weather simulations. The decision as to what kind of seasonal forecast is taken may depend on the forecast quality of the model. For this purpose, it is possible to use an index, for example the Brier score. Below a particular limit below which the added value of the modeled weather forecast is insignificant with respect to long-term climate statistics or multi-year statistics of historic weather data, preference may be given to seasonal weather forecasts based on long-term climate statistics or multi-year statistics of historic weather data.

In addition, the long-term forecast weather progression may follow on, preferably follow on seamlessly, from the short-term forecast weather progression. More particularly, the short-term and long-term forecast weather progression are ascertained in such a way that they can be combined in a time series, preferably in a seamless time series. A seamless transition here means that no discontinuities or other irregularities that do not originate from the weather progression per se and hence are artificial occur in the forecast weather progression, in order to generate forecasts that are as robust and close to reality as possible. For example, the long-term forecast weather progression follows on from the short-term forecast weather progression in such a way that the forecast weather progression has a continuous progression.

In a further embodiment, at least two or more forecast weather sequences or projections for the forecasting period are ascertained on the basis of the weather data provided up to the current date. For example, three forecast weather progressions can be ascertained, with ascertaining of a middle forecast weather progression, an unfavorable forecast weather progression and a favorable forecast weather progression. For instance, using multi-year historic weather data, it is possible to ascertain a typical, for example the most probable, or an average weather progression, for example an average of the weather progressions in a defined period of time, for example the last three, four, five, six, seven, eight, nine, ten years. Alternatively or additionally, on the basis of the recent past, it is possible to ascertain particular seasonal weather forecasts that seem more likely than others. Additionally or alternatively, it is possible that, using multi-year historic weather data, a forecast is made for a weather progression that is favorable—from an agricultural point of view—and/or a weather progression that is unfavorable.

It is further possible to create multiple weather forecasts which preferably cover the spectrum of the weather progressions as have occurred in the past few years. In addition, a probability may be ascertained for the occurrence of each weather progression, such that the weather progressions and the yield estimate resulting therefrom can be compared with one another.

The different weather progression periods (historic, near-future forecast, seasonal forecast, projections) are combined in seamless time sequences (“seamless prediction”). Preferably, the forecast weather progression is ascertained in such a way that there is a seamless transition of the actual weather progression or the weather progression to date and the forecast weather progression. For instance, the actual weather progression or weather progression to date and the forecast weather progression can be combined by a time series with a seamless transition. A seamless transition here means that no discontinuities or other irregularities occur in the combined weather progression, in order to generate forecasts that are as robust and close to reality as possible. For example, the weather progression can be ascertained in such a way that the weather progression to date combined with the forecast weather progression result in a continuous progression. The reference point here for the continuous progression or the occurrence of discontinuities or irregularities may especially be the changeover point between the actual or existing weather progression and forecast weather progression. This should not be considered to include discontinuities or irregularities that result from the weather progression itself, for example in the event of a rapid temperature drop in the event of occurrence of a cold front.

Using multi-year historic weather data, it is possible to achieve a seamless transition by taking account, for example, of such years of historic weather data that have a similar actual or previous weather progression to the previous or actual weather progression up to the current date for the growing period in question. Additionally or alternatively, for model-based or dynamic approaches, it is possible to take account only of those solutions for the forecast weather progression that join seamlessly onto the actual or previous weather progression up to the current date for the growing period in question. Additionally or alternatively, time periods of similar or matching statistics and a similar transition without discontinuities can be put in a series with matching macro weather patterns. The time series of the individual time sections may be generated here in a model-based or dynamic manner. If at least two forecast weather progressions are ascertained, each of the forecast weather progressions is determined in such a way that the actual or previous weather progression up to the current date for the growth period under consideration and the respective forecast weather progression for the forecast period can be combined in a seamless time series.

In a third step S3, a risk of infestation for the forecast period based on the forecast weather progression or multiple risks of infestation each based on the at least two or more weather progression(s) are ascertained. For this purpose, it is additionally possible to use prediction models based, for example, on historic harmful organism data. The historic harmful organism data may comprise satellite data, local image data or scoring that has been recorded for the field under consideration or for an environment in a radius of several kilometers (km), for instance 1 to 10 km, around the field under consideration. The historic harmful organism data may have been transmitted to the external database 24 that can be accessed by the server 12, and directly on the server 12. The historic harmful organism data and the associated prediction models may thus be provided by an external database 24 that can be accessed by the server 12, or directly by the server 12.

In one embodiment, for a forecast of the risk of infestation, one or more digital maps of the field in which the risk of infestation with one or more harmful organisms is drawn or specified in a part-area-specific manner are generated. In this context, part-area-specific refers to a division of the field under consideration into partial areas having different characteristics that affect the risk of infestation. For example, it is conceivable to generate a series of digital maps for a defined harmful organism, for example one map for every month in the year, and to indicate how high is the risk of infestation of the part-area with the harmful organism in the month in question and/or with the forecast weather progression by means of color coding on the maps. For example, the color “red” could mean a risk of infestation of greater than 90%, and the color “green” a risk of infestation of less than 10%. Different yellow and orange shades could be used for the range between 10% and 90%. Other/further modes of representation are conceivable. In a further embodiment, an assessment is made for risks of infestation ascertained as to whether or not a damage threshold has been exceeded.

In a fourth step S4, agricultural measures for the forecast period are optionally ascertained based on the forecast weather progression and/or the forecast risk of infestation. Corresponding different agricultural measures can be ascertained for different forecast weather progressions. If the risk of fungal infestation rises, for example, for a first forecast weather progression at a first date and exceeds the damage threshold at a second date, a spraying measure is ascertained at the second date. If the risk of fungal infestation rises, for example, for a second forecast weather progression at a first date and then falls again owing to the weather conditions, no spraying measure at the second date is ascertained in the case of the second forecast weather progression.

In a fifth step S5, the expected yield of the crop plant at the harvesting date is ascertained on the basis of the forecast weather progression, the forecast risk of infestation and any agricultural measures for the forecast period. It is also possible here to assume at least two or more forecast weather progressions. For instance, it is possible to calculate an expected yield for every weather progression. It is thus possible to generate a decision aid in which the effects of the weather on the risk of infestation and the resulting agricultural measures are forecast with reference to the yield to be expected.

The yields to be expected can be calculated assuming that the forecasts ascertained beforehand are correct (weather progression, occurrence of harmful organisms) and the agricultural measures ascertained are implemented. It is possible here to take account of the fact that there can be an interaction between the occurrence of harmful organisms and the agricultural measures. This is because it can be the aim of an agricultural measure to prevent the occurrence of a forecast harmful organism or to reduce the risk. In such a case, the statement “assuming that the forecasts ascertained beforehand are correct” means that the weather progression occurs as forecast and a risk of the occurrence of harmful organisms does exist as forecast on account of the forecast weather progression, but that the agricultural measures ascertained are implemented and will be successful, which leads to a reduced risk of the occurrence of harmful organisms in relation to the control of harmful organisms (although the risk may also be negligible if the ascertained agricultural has the aim of preventing occurrence of harmful organisms).

The yields to be expected can also be ascertained assuming that the agricultural measures ascertained beforehand are not taken. For instance, the benefit of the agricultural measures ascertained and the effect thereof on the yield to be expected can be made clear.

The ascertained yields to be expected for at least two or more forecast weather progressions, for the correspondingly ascertained risks of infestation and/or the correspondingly ascertained agricultural measures can, for example, be provided and transmitted on the server side in order to be displayed, for example, on the local or mobile computer system 14, 16. For instance, the method can especially be utilized for planning of the upcoming growth period, in order to choose, for example, the seeding date, plan the agricultural measures or predict the planned optimal harvesting date.

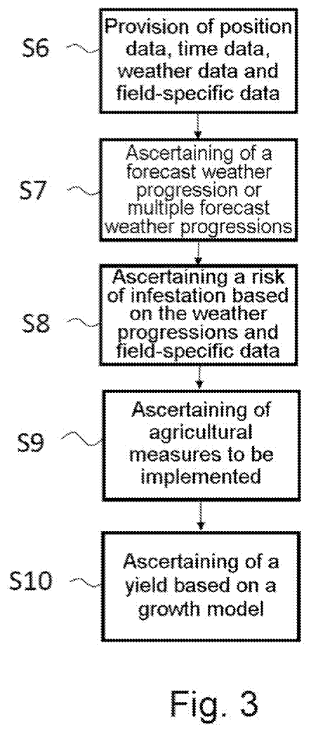

FIG. 3 shows an illustrative method of updating the yields to be expected in the growing of the crop plant with the aid of the localized computer system 10 and especially with the aid of the server 12 of FIG. 1. In particular, the method as shown in FIG. 3 may be implemented after the sowing date and before or after the planned harvesting date. In that case, the current date is after the sowing date in the ongoing growth period and before or after the planned harvesting date of the ongoing growth period. The method can thus especially be utilized for planning during the ongoing growth period or for retrospective assessment after the growth period.

In a first step S6, position data that identify the field and time data that specify the current date and/or the harvesting date, and also field-specific data recorded during the growth period are provided. The position data and time data are provided and utilized as described in association with FIG. 2.

In addition, field-specific data relating to the actual state of the field under consideration are provided. Field-specific data comprise, for example, harmful organism data, measure data and/or growth data. The harmful organism data specify the real progression of the harmful organisms that have actually occurred, the measure data the real progression of the measures that have actually been implemented, and the growth data the real progression of the growth that has actually occurred. Preferably, the field-specific data are recorded as described in connection with FIG. 1. In addition, field-specific data can be provided for the current date and past dates in the ongoing growth period prior to the current date. In addition, it is possible to provide field-specific data from further fields that relate to analogous conditions. Analogous conditions may exist, for example, with regard to sowing, variety, weather conditions, soil or preceding crop.

Moreover, in the first step S6, weather data may be provided for the current date and for past dates in the ongoing growing period before the current date. The weather data provided are provided and utilized as described in association with FIG. 2.

In a second step S7, a forecast weather progression for a forecast period can be ascertained on the basis of the weather data provided up to the current date. The weather progression can be forecast for the ongoing growing period for the crop plants until the harvesting date taking account of the weather progression to date, preferably with a seamless transition of the weather progression to date and the forecast weather progression. The forecast weather progression or the at least two or more forecast weather progressions, as described in association with FIG. 2, is/are ascertained on the basis of the weather data provided at the current date.

In a third step S8, a risk of infestation for the forecast period based on the forecast weather progression or risks of infestation based at least two or more forecast weather progression(s) are ascertained. The risk of infestation is ascertained as described in association with FIG. 2. It is additionally possible here to take account of harmful organism data and/or measure data in order to ascertain the risk of infestation based on the real progression of the harmful organisms that have actually occurred and/or the real progression of the measures that have actually been implemented. In one embodiment, the forecast risk of infestation for the forecast period is ascertained based on the forecast weather progression and based on harmful organism data for the field under consideration. The harmful organism data may comprise, for example, satellite data or image data on the basis of which infestation can be detected. The satellite data may be provided to the server 12 directly via a satellite or indirectly via an external server or an external database 24 that can be accessed by the server 12, or transmitted to the server 12. The image data may be provided to the server 12 with a camera by means of a mobile computer system 16, such as a smartphone or tablet, or transmitted to the server 12. It is also possible here for the harmful organism data to comprise harmful organism data in a radius of several kilometers (km), for instance 1 to 10 km, around the field in question. In addition, the harmful organism data may also comprise those from further fields under analogous conditions. For instance, the risk of infestation may be matched to the real conditions in the growth period.

In a fourth step S9, agricultural measures for the forecast period are ascertained based on the forecast weather progression and/or the ascertained risk of infestation. The agricultural measures are ascertained as described in association with FIG. 2. It is additionally possible here to take account of measure data in order to ascertain agricultural measures for the forecast period on the basis of the measures actually implemented in the course of the growth period to date.

In a fifth step S10, the expected yields in the growing of the crop plants at the harvesting date are ascertained on the basis of the forecast weather progression, the forecast risk of infestation and the agricultural measures. The yields to be expected are ascertained in the fifth step S11, as described in association with FIG. 2. It is additionally possible here to take account of growth data in order to ascertain the yields to be expected on the basis of the real progression of the growth that has actually occurred.

For this purpose, it is possible to use a plant growth model that can be tested and optionally adjusted with reference to the growth data. The plant growth model simulates the growth of a crop of crop plants over a defined period of time. It is also conceivable to use a model based on a single plant that simulates the flows of energy and matter in the individual organs of the plant. Mixed models are additionally usable.

The growth of a crop plant is determined not only by the genetic features of the plant but primarily by the local weather conditions that exist over the lifetime of the plant (quantity and spectral distribution of the insolation, temperature profiles, amounts of precipitation, wind input), the condition of the soil and the nutrient supply.

The crop measures that have already been taken and any infestation with harmful organisms that has occurred can also exert an effect on the plant growth. Growth data, harmful organism data and measure data can thus be taken into account in the growth model.

The following input parameters are preferably included in the modeling:

Weather: daily precipitation totals, total radiation, daily minimum and maximum air temperature, and near-ground temperature and ground temperature, wind speed, inter alia.

Soil: soil type, soil texture, soil nature, field capacity, permanent wilting point, organic carbon, mineral nitrogen content, lodging density, van Genuchten parameters, inter alia.

Crop plant: type, variety, variety-specific parameters, for example specific leaf area index, temperature totals, maximum root depth, inter alia.

Crop measures: seed, sowing date, sowing density, sowing depth, fertilizer, fertilizer volume, number of fertilizing dates, fertilizing date, soil cultivation, harvest residues, crop rotation, distance from the field of the same crop last year, irrigation, inter alia.

The forecast of the evolution of the crop plants grown with time is preferably part-area-specific for the field under consideration.

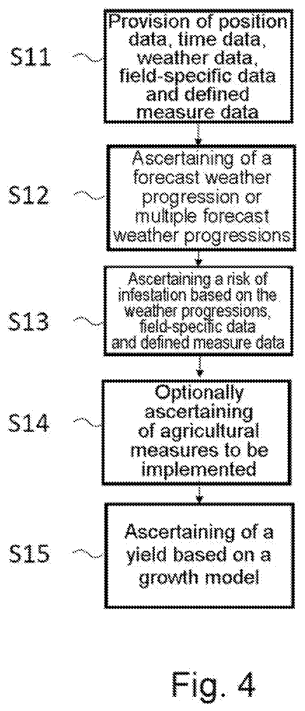

FIG. 4 shows a further illustrative method of ascertaining the yield to be expected in the growing of the crop plant with the aid of the localized computer system 10 in FIG. 1, wherein the yield is ascertained on the basis of predetermined agricultural measures. In particular, the method as shown in FIG. 4 may be implemented before or after the sowing date. In that case, the current date is before or after the sowing date in the ongoing growth period or before or after a planned harvesting date of the ongoing growth period. The method can thus especially be utilized for planning before or during the ongoing growth period and for retrospective assessment of the past growth period.

The method according to FIG. 4 is executed analogously to the methods described in FIGS. 2 and 3 with analogous method steps S11 to S15. By contrast with the methods described in FIGS. 2 and 3, defined measure data that predetermine the agricultural measures are additionally provided in the method shown in FIG. 4. For this purpose, defined measure data may be generated, for instance, in a web-based application on the local or mobile computer system 14, 16 on the basis of a predetermined selection of agricultural measures. The defined measure data may be provided to the server 12. More particularly, the agricultural measures may be specified in a part-area-specific manner for the field under consideration.

If defined measure data are provided in a first step S11, the yields to be expected are ascertained on the basis of the measures predetermined via the defined measure data. If the method of ascertaining the yields to be expected has already been executed at least once for the field in question and/or for the growth period, the measures predetermined from a prior ascertaining of the agricultural measures can be accepted. In addition, agricultural measures may be proposed to a user, for instance, on the client side, for instance from a prior ascertaining of the agricultural measures or from all available agricultural measures. The user can then select agricultural measures, for instance, on the client side. Based on the selection, it is possible to generate defined measure data and transmit them from the local or mobile computer system 14, 16 to the server 12. The method of ascertaining the yields to be expected can then be implemented on the server side based on the measures predetermined.

In addition, the defined measure data can completely or partly specify agricultural measures for the forecast period. If agricultural measures have been predetermined for the complete forecast period or correspondingly defined measure data have been provided, there may be no need for the step of partly or fully ascertaining the agricultural measures. If agricultural measures have been predetermined for a first part of the forecast period or correspondingly defined measure data have been provided, agricultural measures for a second part of the forecast period are ascertained in the method of ascertaining yields to be expected. In this case, the second part of the forecast period is different than the first part. In the second part of the forecast period, in addition, no agricultural measures are predetermined.

The method according to FIG. 4 thus gives the option of ascertaining the yields to be expected for different scenarios relating to the agricultural measures. Thus, the method of the invention, in addition to the scenarios relating to the different forecast weather progressions, enables definition of additional scenarios relating to the agricultural measures. For instance, the cultivation of the field in question before and during the growth period can be simplified. With the aid of the different scenarios and the associated yields to be expected, it is possible to provide a decision aid that enables efficient cultivation of the field in question.

Embodiments of the invention are also

### Embodiment 1: A Method Comprising the Steps of

(A) identifying a field in which crop plants are being grown or are to be grown

(B) providing historic weather data for the field

(C) forecasting a weather progression for the field for the upcoming or ongoing growth period of the crop plants

(D) forecasting pest infestation events for the forecast weather progression

(E) ascertaining agricultural measures for increasing the yield of the crop plants grown

(F) calculating the yields to be expected in the growing of the crop plants assuming that the forecasts mentioned in steps (C) and (D) are correct and the measures ascertained in step (E) are implemented and/or not implemented

(G) displaying the yields to be expected

(H) repeatedly performing steps (C), (D), (E), (F) and (G) taking account of the real progression of the weather up to the respective juncture of performance of the steps, the pest infestation events that have actually occurred and the measures that have actually been implemented,

### Embodiment 2

The method of embodiment 1, in which, in step (C), the historic weather data provided in step (B) are used to generate a weather forecast that constitutes an average weather progression to be expected for the location of the field.

### Embodiment 3

The method of embodiment 1 or 2, in which, in step (C), the historic weather data provided in step (B) are used to generate multiple weather forecasts, one of which leads to a comparatively high harvest yield of the crop plants grown and one of which leads to a comparatively low harvest yield of the crop plants grown.

### Embodiment 4

The method of any of embodiments 1 to 3, in which, in step (C), the historic weather data provided in step (B) are used to create multiple weather forecasts that cover the spectrum of weather progressions as have occurred in the past few years.

### Embodiment 5

The method of any of embodiments 1 to 4, in which, in step (F), the yields to be expected are calculated for every forecast weather progression.

### Embodiment 6

The method of embodiments 1 to 5, in which, in step (D), risks are calculated for the infestation of the field with one or more harmful organisms for every forecast weather progression.

### Embodiment 7

The method of embodiments 1 to 6, in which an agricultural measure in step (E) is a measure for controlling one or more harmful organisms.

### Embodiment 8

The method of embodiments 1 to 7, in which, in step (E), a measure of controlling one or more harmful organisms is ascertained if the risk of infestation with a harmful organism exceeds a damage threshold,

### Embodiment 9: A Computer System Comprising

(A) means of identifying a field in which crop plants are being grown or are to be grown

(B) means of providing historic weather data for the field

(C) means of providing a forecast of a weather progression for the field for the upcoming or ongoing growth period of the crop plants

(D) means of providing a forecast of pest infestation events for the forecast weather progression

(E) means of identifying agricultural measures for increasing the yield of the crop plants grown

(F) means of calculating the yields to be expected in the growing of the crop plants assuming that the forecasts mentioned in steps (C) and (D) are correct and the measures ascertained in step (E) are implemented and/or not implemented

(G) means of displaying the yields to be expected.

### Embodiment 10

a computer program product comprising a computer-readable data storage medium and program code which is stored on the data storage medium and, on execution on a computer system, causes the computer system to execute the following steps:

(A) ascertaining a field in which crop plants are being grown or are to be grown

(B) ascertaining historic weather data for the field

(C) ascertaining a forecast of a weather progression for the field for the upcoming or ongoing growth period of the crop plants

(D) ascertaining a forecast of pest infestation events for the forecast weather progression

(E) ascertaining agricultural measures for increasing the yield of the crop plants grown

(F) calculating the yields to be expected in the growing of the crop plants assuming that the forecasts mentioned in steps (C) and (D) are correct and the measures ascertained in step (E) are implemented and/or not implemented

(G) displaying the yields to be expected

(H) repeatedly performing steps (C), (D), (E), (F) and (G) taking account of the real progression of the weather up to the respective juncture of performance of the steps, the pest infestation events that have actually occurred and the measures that have actually been implemented.

### Embodiment 11

The computer program product of embodiment 10, configured such that a user is able to select and deselect agricultural measures on a display device by actuating an input device and the yield on selection of an agricultural measure is calculated for the case that the selected agricultural measure is implemented, and the yield on deselection of an agricultural measure is calculated for the case that the deselected agricultural measure is not implemented.

### Embodiment 12

The computer program product of embodiment 10 or 11, configured such that the weather progression that has actually occurred at the juncture of utilization of the computer program, the pest infestation events that have actually occurred and the measures that have actually been implemented are included in the calculation of the yields to be expected.

### Embodiment 13

The computer program product of embodiments 10 to 12, configured so as to implement one or more of the methods detailed in claims 1 to 6.

Further embodiments are elucidated in detail hereinafter, without any distinction between the subjects (method, computer system, computer program product). Instead, the elucidations that follow are intended to be analogously applicable to all subjects, irrespective of their context (method, computer system, computer program product).

The term “field” is understood to mean a spatially delimitable region of the surface of the Earth which is in agricultural use by planting of crop plants in such a field, supplying them with nutrients and harvesting them.

The term “crop plant” is understood to mean a plant that is purposely grown as a useful or ornamental plant through human intervention.

In a first step, the field in which crop plants are being grown or are to be grown, and which is considered in detail in the course of the method of the invention, is identified.

Typically, the identification is effected using geocoordinates that unambiguously determine the location of the field. The present method is typically executed with the aid of a computer program installed on a computer system. Typically, the geocoordinates of the field are therefore transferred into the computer program. For example, a user of the computer program could input the geocoordinates via a keyboard. It is also conceivable that the user of the computer program views geographic maps on a computer screen and marks the boundaries of the field under consideration on such a map, for example with a computer mouse. The identification of the field accordingly fixes the region of the earth's surface that is considered in the further course of the method. In a further step, historic weather data are provided for the field. Historic weather data are provided, for example, by commercial suppliers. With reference to the historic weather data, in a further step, a forecast is made for the progression of the weather for the upcoming or ongoing growth period. Whether a weather forecast is created for the upcoming growth period of the crop plants to be grown in the field or for the ongoing growth period of the crop plants being grown in the field depends on the juncture at which the forecast is made: before commencement of the upcoming growth period or after commencement of the growth period. It is conceivable that multiple forecasts are made. It is conceivable that historic weather data are used to ascertain a typical, i.e. average, weather progression. It is conceivable that, in addition, the historic weather data are used to make a forecast for a weather progression that is comparatively favorable—from an agricultural point of view—and/or a weather progression that is comparatively unfavorable. The aim in the forecasting of the weather progression may be to forecast weather with maximum precision for the upcoming or ongoing growth period. As is well known, the weather can be forecast comparatively accurately for the next few days, whereas weather forecasts for a date in a few weeks or months in the future are comparatively inexact. For periods in which the weather can be forecast only inexactly, therefore, historic weather data are of good suitability, in order to use trends that have frequently been observed in the past few years as a basis for the forecast of the future weather. In a preferred embodiment, multiple weather forecasts are created, which preferably cover the spectrum of the weather progressions as have occurred in the past few years. In a preferred embodiment, a probability is also ascertained and reported for the occurrence of each weather progression, such that the weather progressions can be compared with one another. For every forecast weather progression, in a further step of the present method, a forecast is made for the occurrence of one or more pest infestations. Preferably, the forecast ascertains risks of infestation for one or more harmful organisms. A “harmful organism” is understood to mean an organism that can appear in the growing of crop plants and can damage the crop plant, adversely affect the harvest of the crop plant or compete with the crop plant for natural resources. Examples of such harmful organisms are weed plants, weed grasses, animal pests, for example beetles, caterpillars and worms, fungi and pathogens (e.g. bacteria and viruses). Even though viruses are not among the organisms from a biological point of view, they shall nevertheless be covered here by the term “harmful organism”. The term “weed plant” (plural: weed plants) is understood to mean plants from spontaneous accompanying vegetation (segetal flora) in crop plant crops, grassland or gardens that are not deliberately planted there and develop, for example, from the seed potential in the soil or by aerial transmission. The term is not limited to weeds in the actual sense, but also includes grasses, ferns, mosses or woody plants. In the crop protection sector, the term “weed grass” (plural: weed grasses) is frequently also utilized in order to illustrate a delimitation from the herbaceous plants. In the present text, the term “weed” is used as an umbrella term that is intended to include the term “weed grass”. For the forecasting of a pest infestation, it is possible, for example, to use prediction models described in the prior art. The commercially available decision support system “expert”, for prediction of a pest infestation, uses data relating to the crop plants being grown or to be grown (stage of development, growth conditions, crop protection measures), relating to weather conditions (temperature, hours of sunshine, wind speed, precipitation) and relating to the known pests/diseases (limits of economic viability, seedling/disease pressure) and calculates a risk of infestation on the basis of these data (Newe M., Meier H., Johnen A., Volk T.: proPlant expert.com—an online consultation system on crop protection in cereals, rape, potatoes and sugarbeet. EPPO Bulletin 2003, 33, 443-449; Johnen A., Williams I. H., Nilsson C., Klukowski Z., Luik A., Ulber B.: The proPlant Decision Support System: Phenological Models for the Major Pests of Oilseed Rape and Their Key Parasitoids in Europe, Biocontrol-Based Integrated Management of Oilseed Rape Pests (2010) Ed.: Ingrid H. Williams. Tartu 51014, Estonia. ISBN 978-90-481-3982-8. p. 381-403; www.proPlantexpert.com). For forecasting of the pest infestation events, it is also possible to take account of actual pest infestation events in the past. Preferably, risks of infestation are ascertained for those pests that have occurred in the past in the field in question and/or adjacent fields. The risks of infestation are preferably ascertained in a part-area-specific manner. It is conceivable, for example, that some part-areas of the field, owing to their location, are particularly frequently and/or particularly significantly affected by a pest infestation and/or that the infestation with a harmful organism frequently emanates from one or more defined part-areas. In a preferred embodiment, for a forecast of the weather progression, one or more digital maps of the field in which the risk of infestation with one or more harmful organisms is drawn in a part-area-specific manner are generated. For example, it is conceivable to generate a series of digital maps for a defined pest, for example one map for every month in the year, and to indicate how high is the risk of infestation of the part-area with the pest in the month in question and with the forecast weather progression by means of color coding on the maps. For example, the color “red” could mean a risk of infestation of greater than 90%, and the color “green” a risk of infestation of less than 10%. Different yellow and orange shades could be used for the range between 10% and 90%. Other/further modes of representation are conceivable. In a preferred embodiment, an assessment is made for risks of infestation ascertained as to whether or not a damage threshold has been exceeded. “Damage threshold” is a term from agriculture, forestry and horticulture. It indicates the infestation density with pathogens or diseases or infestation with weeds from which control is economically viable. Up to this value, extra economic expenditure through control is greater than the harvest failure of which there is a risk. If the infestation or weed pressure exceeds this value, the control costs are at least compensated for by the extra yield to be expected. According to the nature of a pest or disease, the damage threshold may be very different. In the case of pests or diseases that can be controlled only with high expenditure and adverse accompanying effects on further production, the damage threshold can be very high. If, however, even a small infestation can become a propagation source that threatens to destroy the entire production, the damage threshold may be very low. There are many examples in the prior art relating to the ascertaining of damage thresholds (see, for example, Claus M. Brodersen: Informationen in Schadschwellenmodellen, Reports from the GIL, volume 7, pages 26 to 36, http://www.gil-net.de/Publikationen/7_26.pdf). In a further step, agricultural measures for increasing the yield of the crop plants being grown are ascertained. The term “agricultural measure” is understood to mean any measure in the field for crop plants that is necessary or economically viable and/or environmentally advisable in order to obtain a plant product. Examples of agricultural measures are: soil cultivation (e.g. ploughing), deploying the seed (sowing), irrigation, removal of weed plants/weed grasses, fertilizing, control of harmful organisms, harvesting. Preferably, the agricultural measures are measures for controlling the forecast pest infestation events. The measures are ascertained especially by the selection of a suitable crop protection product, the fixing of dates when the crop protection product should be applied, and fixing of the amount of crop protection product to be applied. The measures are preferably ascertained in a part-area-specific manner. The term “crop protection product” is understood to mean a composition that serves to protect plants or plant products from harmful organisms or to prevent their effect, to destroy unwanted plants or plant parts, to inhibit unwanted growth of plants or to prevent such growth and/or to influence the life processes of plants in a different manner than nutrients. Examples of crop protection products are herbicides, fungicides and pesticides (for example insecticides). Preference is given to ascertaining those measures that have a maximum cost/benefit ratio. The ascertaining of measures preferably takes account of legal aspects and environmental protection aspects. For example, it is conceivable that a selected crop protection product may be applied only at particular dates and/or in particular maximum amounts. These and similar restrictions are preferably taken into account in the ascertaining of the measures. In a further step, the yields to be expected when the crop plants are grown under the conditions of the scenarios under consideration are ascertained. For this purpose, a plant growth model may be used. The term “plant growth model” is understood to mean a mathematical model that describes the growth of a plant as a function of intrinsic (genetic) and extrinsic (environmental) factors. Plant growth models exist for a multitude of crop plants. An introduction into the creation of plant growth models is given, for example, by the books i) “Mathematische Modellbildung and Simulation” by Marco Gunther and Kai Velten, published by Wiley-VCH Verlag in October 2014 (ISBN: 978-3-527-41217-4), and ii) “Working with Dynamic Crop Models” by Daniel Wallach, David Makowski, James W. Jones and Francois Brun, published in 2014 in Academic Press (Elsevier), USA. The plant growth model typically simulates the growth of a crop of crop plants over a defined period of time. It is also conceivable to use a model based on a single plant that simulates the flows of energy and matter in the individual organs of the plant. Mixed models are additionally usable. The growth of a crop plant is determined not only by the genetic features of the plant but primarily by the local weather conditions that exist over the lifetime of the plant (quantity and spectral distribution of the insolation, temperature profiles, amounts of precipitation, wind input), the condition of the soil and the nutrient supply. The crop measures that have already been taken and any infestation with harmful organisms that has occurred can also exert an effect on the plant growth and can be taken into account in the growth model. The plant growth models are generally what are called dynamic process-based models (see “Working with Dynamic Crop Models” by Daniel Wallach, David Makowski, James W. Jones and Francois Brun, published in 2014 in Academic Press (Elsevier), USA), but may also be entirely or partly rule-based or statistical or data-supported/empirical. The models are generally what are called point models. The models here are generally calibrated such that the output reflects the spatial representation of the input. If the input has been ascertained at a point in space or is interpolated or estimated for a point in space, it is generally assumed that the model output is applicable to the whole adjacent field. Application of what are called point models calibrated at the field level to wider, generally rougher scales is known (see, for example, H. Hoffmann et al.: Impact of spatial soil and climate input data aggregation on regional yield simulations, 2016, PLoS ONE 11(4): e0151782. doi:10.1371/journal.pone.0151782). Application of this so-called point model to multiple points within a field enables part-area-specific modeling here. However, spatial dependences are neglected here, for example in the groundwater budget. On the other hand, there also exist systems for time/space-explicit modeling. Spatial dependences are taken into account here. Examples of dynamic, process-based plant growth models are Apsim, Lintul, Epic, Hermes, Monica, STICS inter alia. The following input parameters are preferably included in the modeling:

Weather: daily precipitation totals, total radiation, daily minimum and maximum air temperature, and near-ground temperature and ground temperature, wind speed, inter alia.

Soil: soil type, soil texture, soil nature, field capacity, permanent wilting point, organic carbon, mineral nitrogen content, lodging density, van Genuchten parameters, inter alia.

Crop plant: type, variety, variety-specific parameters, for example specific leaf area index, temperature totals, maximum root depth, inter alia.

Crop measures: seed, sowing date, sowing density, sowing depth, fertilizer, fertilizer volume, number of fertilizing dates, fertilizing date, soil cultivation, harvest residues, crop rotation, distance from the field of the same crop last year, irrigation, inter alia.

The forecast of the evolution of the crop plants grown with time is preferably part-area-specific. The calculation of the yields to be expected is made assuming that the forecasts ascertained beforehand are correct (weather progression, pest infestation events). The calculation of the yields to be expected is also made assuming that the agricultural measures ascertained beforehand are taken and/or not taken. It is conceivable that the user of the computer program product can study the effect of the measures on the yields to be expected on a computer by, for example, deselecting recommended measures and then the computer program calculates how the yield changes if the measure deselected is not implemented. Measures are preferably selected and deselected in a part-area-specific manner. The yields to be expected are displayed to a user on a display device. The display device is typically a screen which is part of the present computer system. Preferably, the yield to be expected is indicated for individual part-areas and/or the entire field. The display may be graphic-assisted, for example with the aid of bar diagrams or the like. The user is thus able to view various scenarios on the computer and see what yields are the result if a particular forecast weather progression is actually realized and/or what yields are the result if particular measures are taken or not taken. Preferably, the yields to be expected are displayed in a part-area-specific manner in the form of digital maps on the computer. In a further step, the steps (C), (D), (E), (F) and (G) mentioned are repeated, taking account of the progression of the weather up to the respective juncture of implementation of the steps, pest infestation events that have actually occurred and actually agricultural measures. The present computer program product is preferably configured such that it is automatically updated. Updating means that the weather progression that has actually occurred up to the juncture of the respective update, the pest infestation events that have actually occurred and the measures that have actually been implemented (for example for control of pest infestation events) are included in the calculation of yields to be expected. The updating can be effected automatically, for example, whenever the user starts or calls up the computer program. It is alternatively conceivable that the update is effected at a fixed time, for example every day or every week. It is alternatively conceivable that an update is effected at irregular intervals, for example whenever there is a significant deviation of the real conditions from those forecast. In the event of an update, the steps (C), (D), (E), (F) and (G) detailed above are repeated. Assuming that the user has executed the present computer program product on a first occasion at a first juncture and the yields can be calculated for a forecast weather progression and on the condition that the measures recommended from step (E) are actually taken. At a later, second juncture, the user calls up the present computer program product again. In the intervening period, there was a defined weather progression that affects the plant growth of the crop plants grown and/or the risk of pest infestation. The present computer program product ascertains the actual weather progression and adjusts the forecast for the risk of pest infestation to the actual weather progression. In addition, one or more updated weather forecasts are created and the corresponding risks of pest infestation are likewise updated. On the basis of the updated risks of pest infestation, new measures for controlling the pests are ascertained. Finally, an updated yield to be expected is calculated and displayed.

1. A method of ascertaining yields to be expected in the growing of crop plants with the aid of a computer system (10), comprising the steps of
(A) identifying (S1, S6, S11) a field in which crop plants are being grown or are to be grown
(B) forecasting (S2, S7, S12) a weather progression for the field for the upcoming or ongoing growing period for the crop plants until the planned harvest taking account of the weather progression to date,
(C) forecasting (S3, S8, S13) for the occurrence of one or more harmful organisms in the field for the forecast weather progression
(D) ascertaining (S4, S9, S14) agricultural measures for the upcoming or ongoing growing period of the crop plants until the planned harvest
(E) calculating (S5, S10, S15) the yield to be expected in the growing of the crop plants assuming that the forecasts from steps (B) and (C) are correct and the measures ascertained in step (D) are implemented
(F) displaying the yield to be expected
(G) repeatedly performing steps (B), (C), (D), (E) and (F) taking account of the real progression of the weather up to the respective juncture of performance of the steps, the harmful organisms that have actually occurred and the measures that have actually been implemented, with provision for step (B) of weather data relating to an existing progression of the weather and, for steps (C), (D), (E), recorded field-specific data, especially harmful organism data relating to harmful organisms that have actually occurred, growth data relating to the real progression of the growth that has actually occurred and/or measure data relating to measures that have actually been implemented.

2. The method according to claim 1, wherein measure data that at least partly define agricultural measures for the upcoming or ongoing growth period are provided and, in step (E), the expected yield is ascertained on the basis of the defined measures that may have been ascertained in step (D).

3. The method according to claim 1, wherein at least two different weather progressions are forecast in step (B), and steps (C), (D), (E), (F) are performed for each of the at least two different weather progressions.

4. The method according to claim 1, wherein the yield to be expected is calculated in step (E) for the case that one or more ascertained agricultural measure(s) is/are not implemented.

5. The method according to claim 1, wherein, in step (E), a dynamic, process-based plant growth model is used.

6. The method according to claim 1, wherein, in step (E), a plant growth model that ascertains the growth of a crop of crop plants or of a single plant over a defined period of time is used.

7. The method according to claim 1, wherein, in step (E), a plant growth model that takes account not only of genetic features of the crop plant but also of the local weather conditions that exist over the lifetime of the crop plant, the condition of the soil and the nutrient supply is used.

8. The method according to claim 1, wherein, in step (E), a plant growth model that takes account of agricultural measures taken and infestation with harmful organisms is used.

9. The method according to claim 1, wherein in-model parameters or calculated state variables are adjusted in the repeated performance as per step (G).

10. The method according to claim 1, wherein the field-specific data comprise at least one of the following parameters:
vegetation index (e.g. NDVI) or leaf area index (LAI),
infections actually observed in the plant crop,
growth stages or maturity of the crop actually observed,
agricultural measures actually taken,
environmental variables actually observed.

11. A computer system (10, 12) comprising
(A) means (26) of identifying a field in which crop plants are being grown or are to be grown
(B) means (28) of providing a forecast of a weather progression for the field for the upcoming or ongoing growing period for the crop plants until the planned harvest taking account of the weather progression to date,
(C) means (30) of providing a forecast for the occurrence of one or more harmful organisms in the field for the forecast weather progression
(D) means (32) of identifying agricultural measures for the upcoming or ongoing growing period of the crop plants until the planned harvest
(E) means (34) of calculating the yield to be expected in the growing of the crop plants assuming that the forecasts from steps (B) and (C) are correct and the measures ascertained in step (D) are implemented
(F) means (26) of displaying the yield to be expected
wherein the computer system is configured such that it repeatedly performs steps (B), (C), (D), (E) and (F) taking account of the real progression of the weather up to the respective juncture of performance of the steps, the harmful organisms that have actually occurred and the measures that have actually been implemented, with provision for step (B) of weather data relating to an existing progression of the weather and, for steps (C), (D), (E), recorded field-specific data, especially harmful organism data relating to harmful organisms that have actually occurred, growth data relating to the real progression of the growth that has actually occurred and/or measure data relating to measures that have actually been implemented.

12. The computer system (10, 12) according to claim 11, comprising
an input unit (26) by means of which data and control commands can be input into the computer system, where the computer system is configured such that a user can specify a field by means of the input unit and can input information about the crop plants being grown or to be grown in the field,
a receiver unit (26) for receiving weather forecasts for the specified field,
a database (24) with information relating to the crop plants being grown or to be grown
a processing unit (34) configured so as to be able to calculate probabilities of the occurrence of one or more harmful organisms on the basis of weather forecasts, and configured so as to be able to call up agricultural measures from the database for the crop plants being grown or to be grown, and configured so as to be able to calculate expected yields for the crop plant on the basis of the forecast weather progressions, harmful organisms and agricultural measures ascertained, and a display device (14, 16) on which the yield forecasts can be displayed to a user.

13. A computer program product comprising a computer-readable data storage medium and program code which is stored on the data storage medium and, on execution on a computer system, causes the computer system to execute the following steps:
(A) ascertaining a field (S1, S6, S11) in which crop plants are being grown or are to be grown
(B) ascertaining a weather progression (S2, S7, S12) for the field for the upcoming or ongoing growing period for the crop plants until the planned harvest taking account of the weather progression to date,
(C) ascertaining a forecast (S3, S8, S13) for the occurrence of one or more harmful organisms in the field for the forecast weather progression
(D) ascertaining agricultural measures (S4, S9, S14) for the upcoming or ongoing growing period of the crop plant until the planned harvest
(E) calculating the yield to be expected (S5, S10, S15) in the growing of the crop plants assuming that the forecasts from steps (B) and (C) are correct and the measures ascertained in step (D) are implemented
(F) displaying the yield to be expected
(G) repeatedly performing steps (B), (C), (D), (E) and (F) taking account of the real progression of the weather up to the respective juncture of performance of the steps, the harmful organisms that have actually occurred and the measures that have actually been implemented, with provision for step (B) of weather data relating to an existing progression of the weather and, for steps (C), (D), (E), recorded field-specific data, especially harmful organism data relating to harmful organisms that have actually occurred, growth data relating to the real progression of the growth that has actually occurred and/or measure data relating to measures that have actually been implemented.

14. A computer program product configured so as to implement one or more of the methods detailed in claim 1.

15. The computer program product according to claim 13, configured such that a user is able to select and deselect agricultural measures on a display device by actuating an input device and the yield on selection of an agricultural measure is calculated for the case that the selected agricultural measure is implemented, and the yield on deselection of an agricultural measure is calculated for the case that the deselected agricultural measure is not implemented.
